(19) United States
(12) Patent Application Publication
(10) Pub. No.: US 2025/0361560 A1
(43) Pub. Date: Nov. 27, 2025

(54) METHODS FOR DETERMINING MITOCHONDRIAL DNA DAMAGE

(71) Applicant: UNIVERSITY OF NEWCASTLE UPON TYNE, Tyne (GB)

(72) Inventors: Mark Anthony BIRCH-MACHIN, Tyne (GB); Roisin STOUT, Tyne (GB)

(21) Appl. No.: 18/851,659

(22) PCT Filed: Mar. 30, 2023
(86) PCT No.: PCT/GB2023/050834

§ 371 Date: Sep. 26, 2024

(30) Foreign Application Priority Data
Mar. 30, 2022 (GB) 2204595.9

(51) Int. Cl. C12Q 1/6883

(52) U.S. Cl. CPC C12Q 1/6883; C12Q 2600/136; C12Q 2600/16

(57) ABSTRACT

The disclosure relates to methods of determining the level of mitochondrial DNA (mtDNA) damage in a cell population (for example a skin cell population). The invention further relates to methods of determining the ability of a test agent to prevent or repair mtDNA damage in a cell population, as well as methods for monitoring progression of mtDNA damage in a cell population. The invention also relates to kits for use in the methods of the invention, as well as use of a mtDNA fragment that the inventors have identified is especially susceptible to damage caused by environmental factors, such as UVR and/or pollution.

### FIELD OF INVENTION

The present invention relates to methods of determining the level of mitochondrial DNA (mtDNA) damage in a cell population (for example a skin cell population). The invention further relates to methods of determining the ability of a test agent to prevent or repair mtDNA damage in a cell population, as well as methods for monitoring progression of mtDNA damage in a cell population. The invention also relates to kits for use in the methods of the invention, as well as use of a mtDNA fragment that the inventors have identified is especially susceptible to damage caused by environmental factors, such as UVR and/or pollution.

### BACKGROUND

Mitochondria play a central role in cellular energy provision. These organelles contain their own genome with a modified genetic code. The human mitochondrial DNA (mtDNA) is a double-stranded, circular molecule of 16,569 base pairs (bp) and contains 37 genes coding for two rRNAs, 22 tRNAs and 13 proteins. The mtDNA-encoded proteins are all subunits of enzyme complexes of the oxidative phosphorylation system. This process is performed by means of electron flow between four enzymes, of which three are proton pumps, in the inner mitochondrial membrane.

MtDNA is highly susceptible to oxidative damage because it is not compacted around histones and is localized near the electron transport chain, which is a major source of reactive oxygen species (ROS). In addition, mtDNA has few noncoding regions, increasing the chances of mutagenicity in coding regions. Furthermore, mitochondria are highly enriched in iron microenvironments, thus favouring the formation of OH that, due to its short half-life, preferentially reacts with mitochondrial components, including mtDNA, resulting in mtDNA damage.

In the skin, mitochondria are even more susceptible to oxidative damage due to being continuously exposed to external stressors, such as ultraviolet radiation (UVR), and/or pollutants, for example urban dust. Exposure to external stress such as UVR from the sun can increase a person's risk of developing skin cancers, and accelerate the appearance of signs of skin aging, such as loss of skin elasticity, wrinkling and hyperpigmentation.

This is why methods that can reliably quantify and monitor mtDNA damage (especially UVR or pollution induced damage) are needed. The present invention aims to provide such methods.

### SUMMARY OF THE INVENTION

The present invention is based on the inventors' identification of a specific mtDNA region that is particularly susceptible to oxidative damage caused by environmental stressors, such as UVR exposure. The inventors tested sixteen different regions of mtDNA and surprisingly found that one particular region is highly sensitive to damage caused by external stressors, such as UVR. This region is located between nucleotides from 4512 to 6969. However, even more surprisingly, the inventors found that different parts within this region have a different susceptibility to damage depending on whether exposure to the external stressor (such as UVR) is chronic or acute.

Specifically the inventors found that the region of mtDNA located between nucleotides from 5741 to 6969 is more susceptible to a high dose of UVR exposure over a short period of time, similar to the amount of UVR that may result in sunburn. The region of mtDNA located between nucleotides from 4512 to 5744, on the other hand, was found to be more susceptible to damage as a result of lower doses of UVR exposure over a longer period of time, simulating the amount of exposure a person would typically have in a day. The inventors also found that surprisingly, mtDNA damage that is caused by the simulation of daily doses of UVR exposure, underwent almost complete repair within about 24 hours post exposure, whereas mtDNA damage caused by the simulation of sunburn did not. Further, surprisingly, the inventors found that fragments bigger in size (i.e. fragments of about 1000 bases or more) are more useful for determining mtDNA repair about 24 hours post exposure as opposed to smaller fragments (i.e. fragments of about 650 bases or less). These findings have led the inventors to the development of the various aspects of the present invention.

In addition to studying the effects of UVR on these mtDNA regions, the inventors also investigated whether these regions could be used to monitor mtDNA damage induced by pollution (such as urban dust) and found that these regions may indeed be used to detect mtDNA damage caused by pollution, as shown in Example 2 of the present disclosure.

Accordingly, in a first aspect, the present invention provides a method of determining the level of mitochondrial DNA (mtDNA) damage in a cell population, the method comprising:
a) quantifying the total amount of mtDNA in a sample of the cell population;
b) quantifying the amount of a mtDNA fragment comprising at least 200 bases from a region of mtDNA located between nucleotides from 4412 to 7069 in the sample of the cell population; and
c) comparing the amount of said fragment to the total amount of mtDNA in the sample, and thereby determining the level of mtDNA damage in the cell population.

In a further aspect, the present invention provides a method of determining the ability of a test agent to prevent or repair mtDNA damage in a cell population, the method comprising:
a) determining the level of mtDNA damage in a first sample of the cell population, wherein the level of mtDNA damage is determined by a method of the first aspect;
b) providing the test agent to the cell population;
c) determining the level of mtDNA damage in a second sample of the cell population, wherein the level of mtDNA damage is determined by a method of the first aspect; and
d) comparing the level of mtDNA damage determined in step c) to the levels of mtDNA damage determined in step a); wherein
   i) no change between the level of mtDNA damage determined in step c) as compared to step a) is indicative of the test agent having the ability to prevent mtDNA damage; or
   ii) a decrease between the level of mtDNA damage determined in step c) as compared to step a) is indicative of the test agent having the ability to repair mtDNA damage.

In a further aspect, the present invention provides a method of monitoring progression of mtDNA damage in a cell population, the method comprising:
a) determining the level of mtDNA damage in a first sample of the cell population, wherein the level of mtDNA damage is determined by a method of the first aspect;
b) determining the level of mtDNA damage in a second sample of the cell population wherein the level of mtDNA damage is determined by a method of the first aspect, and wherein the second sample has been obtained from the cell population at a later time point than the first sample; and
c) comparing the amount of mtDNA damage determined in step b) as compared to step a); wherein an increase in mtDNA damage in step b) as compared to step a) is indicative of progression of mtDNA damage.

Suitably, the cell population may be a skin cell population.

Suitably, the mtDNA damage may be caused by oxidative stress.

Suitably, the oxidative stress may be caused by exposure to UVR and/or exposure to a pollutant (such as urban dust).

Suitably, the fragment may be from a region of mtDNA located between nucleotides from 4512 to 6969.

Suitably, the fragment from a region of mtDNA located between nucleotides from 4512 to 6969 may comprise at least about 200 bases, at least about 500 bases, at least about 650 bases, at least about 1000 bases, at least about 1200 bases, at least about 1600 bases, at least about 2000 bases, at least about 2400 bases.

Suitably, the fragment may comprise or consist of 2458 bases.

Suitably, the exposure to UVR may be chronic and/or exposure to a pollutant (such as urban dust) may be acute.

Suitably, when exposure to UVR is chronic and/or exposure to a pollutant is acute, the fragment may be from a region of mtDNA located between nucleotides from 4512 to 5744.

Suitably, the fragment from a region of mtDNA located between nucleotides from 4512 to 5744 may comprise at least about 200 bases, at least about 500 bases, at least about 650 bases, at least about 1000 bases, or at least about 1200 bases.

Suitably the fragment may comprise or consist of 1233 bases.

Suitably, the exposure to UVR may be acute and/or exposure to a pollutant may be chronic.

Suitably, when exposure to UVR is acute and/or exposure to a pollutant is chronic, the fragment may be from a region of mtDNA located between nucleotides from 5741 to 6969.

Suitably, the fragment from a region of mtDNA located between nucleotides from 5741 to 6969 may comprise at least about 200 bases, at least about 500 bases, at least about 650 bases, at least 1000 bases, or at least 1200 bases.

Suitably, the fragment may comprise or consist of 1229 bases.

Suitably, the step of quantifying the total amount of mtDNA may comprise amplifying a damage resistant mtDNA region.

Suitably, the damage resistant mtDNA region may be a fragment consisting of 100 bases or less.

Suitably, the damage resistant mtDNA region may be a fragment consisting of 83 bases or less, optionally located between nucleotides from 16042 to 16124.

Suitably, the step of quantifying the amount of a mtDNA fragment may comprise amplifying the fragment.

Suitably the step of amplifying the damage resistant mtDNA region and/or fragment may be by quantitative PCR (qPCR).

In a further aspect, the present invention provides a kit for determining the level of mitochondrial DNA (mtDNA) damage in a cell population, the kit comprising a primer set for amplifying a mtDNA fragment comprising at least about 200 bases from a region of mtDNA located between nucleotides from 4412 to 7069.

Suitably, the kit may comprise a primer set for amplifying a mtDNA fragment comprising at least about 200 bases from a region of mtDNA located between nucleotides from 4512 to 6969.

Suitably, the primer set may comprise the nucleic acid sequences selected from the group consisting of: (1) SEQ ID NO: 4 and SEQ ID NO: 7; (2) SEQ ID NO: 4 and SEQ ID NO: 5; (3) SEQ ID NO: 6 and SEQ ID NO: 7; (4) SEQ ID NO: 10 and SEQ ID NO: 11; (5) SEQ ID NO: 12 and SEQ ID NO: 13; and (5) SEQ ID NO: 14 and SEQ ID NO: 15.

Suitably, the kit may comprise a primer set for amplifying a damage resistant mtDNA region.

Suitably, the primer set for amplifying a damage resistant mtDNA region may comprise the nucleic acid sequences as shown in SEQ ID NO: 8 and SEQ ID NO:9.

In a further aspect, the present invention provides a use of a mtDNA fragment comprising at least about 200 bases from a region of mtDNA located between nucleotides from 4412 to 7069 for determining or monitoring the level of mtDNA damage.

Suitably, the fragment may be between nucleotides from 4512 to 6969.

Suitably, the fragment may comprise at least about 200 bases, at least about 500 bases, at least about 650 bases, at least about 1000 bases, at least about 1200 bases, at least about 1600 bases, or at least about 2000 bases.

It will be recognised that, except where the context requires otherwise, embodiments described in respect of one aspect of the invention, for example a method of determining the level of mtDNA damage, or a method of monitoring the level of mtDNA damage, or a method of determining the ability of a test agent to repair or prevent mtDNA damage, will be applicable to all of the aspects of the invention. Thus, for example, embodiments mentioned in the context of one method of the invention are also applicable to other methods, as well as kits and uses of the invention.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects and embodiments of the invention are described in further detail below.

### BRIEF DESCRIPTION OF THE FIGURES

Embodiments of the invention are further described hereinafter with reference to the accompanying drawings, in which:

FIG. 1 is a schematic diagram of a mtDNA molecule showing the 16 regions that were tested for susceptibility to UVR damage. Regions 6 and 7 were found to be most susceptible to UVR damage.

FIG. 2 is a graph showing the effects of acute and chronic UVR exposure on different regions of mtDNA. Region 6 (located between nucleotides from 4512 to 5744) and 7 (located between nucleotides from 4741 to 6969) was found to be most susceptible to UVR induced mtDNA damage.

FIG. 3 are graphs showing the effects of acute (sunburn) or chronic (daily doses) UVR exposure on mtDNA damage in regions 6 and 7 individually or when combined.

FIG. 4 are graphs showing effects of chronic UVR exposure on mtDNA damage determined using different size fragments. It can be seen from FIG. 4A that fragments as small as 218 bases (located between nucleotides from 6067 to 6284), 495 bases (located between nucleotides from 6067 to 6561), and 648 bases (located between nucleotides from 4619 to 5266) can be used to detect the amount of mtDNA damage. FIG. 4A shows mtDNA damage immediately upon exposure to chronic UVR as compared to non-UVR exposed control cells.

FIG. 4B shows that for smaller fragments, such as fragments having about 650 bases or less, it may be more desirable to measure mtDNA damage 24 hours post exposure to UVR. The results in FIGS. 4A and B are based on n=2. Accordingly, where the changes are not statistically significant, the inventors believe that this is due to a small sample size, and increasing the sample size would render the results statistically significant.

FIG. 5 graphs showing effects of urban dust on mtDNA damage. FIGS. 5A and B show 2^{ddCt} values for increasing concentrations of urban dust at 8 hours with 1233 bp and 1229 bp qPCR assays, respectively. HDFn cells were treated with 5, 10, 25, 50 and 100 ug/ml urban dust and 2.16 SED, and mtDNA damage was assessed using the 1233 bp (A.) and 1229 bp (B.) assays after 8 hours. Data are presented as mean+SD (n=3) and are compared to the 0 μg/ml urban dust control (shown by dotted line). Statistical difference was determined using a One-Way ANOVA with Dunnett's multiple comparisons test. All groups were compared to the control group. * p<0.05. Figures C and D show 2^{ddct} values for increasing concentrations of urban dust at 24 hours with 1233 bp and 1229 bp qPCR assays, respectively (corresponding to regions 6 and 7 respectively as described in FIG. 1 and used in FIGS. 2-4). HDFn cells were treated with 5, 10, 25, 50 and 100 ug/ml urban dust and 2.16 SED, and mtDNA damage was assessed using the 1233 bp (region 6 primers) (C.) and 1229 bp (region 7 primers) (D.) assays after 24 hours. Data are presented as mean+SD (n=3) and are compared to the 0 ug/ml urban dust control (shown by dotted line). Statistical difference was determined using a One-Way ANOVA with Dunnett's multiple comparisons test. All groups were compared to the control group. * p<0.05.

### DETAILED DESCRIPTION

Methods for Determining the Levels of mtDNA Damage, Methods for Monitoring mtDNA Damage, and Methods for Determining a Test Agent's Ability to Prevent or Repair mtDNA Damage

In one aspect, the present invention provides a method of determining the level of mitochondrial DNA (mtDNA) damage in a cell population.

As used herein the term “mtDNA damage” refers to a loss of integrity of the mitochondrial genome. Suitably the loss of integrity may be observed by a single or double stranded break in the mitochondrial genome. Suitably the single or double stranded break is at a region of mtDNA located between nucleotides from 4412 to 7069, more suitably a region located between nucleotides from 4512 to 6969. Thus, in the context of the present disclosure, a mtDNA molecule having a single or double stranded break in the region located between nucleotides from 4412 to 7069 (for example between nucleotides from 4512 to 6969) may be referred to herein as a “damaged mtDNA molecule”. By the same token, a mtDNA molecule that does not have a single or double stranded break in the region located between nucleotides from 4412 to 7069 (for example between nucleotides from 4512 to 6969) may be referred to herein as a “healthy mtDNA molecule”.

Suitably, mtDNA damage may be caused by oxidative stress. As used herein, the term “oxidative stress” refers to pathophysiological effects of reactive oxygen species (ROS) on normal cellular structure and/or function. ROS is a term that collectively describes molecules that have a reactive oxygen moiety. Examples of ROS include hydroxyl radicals and/or superoxide. Oxidative stress may cause damage to DNA, RNA, proteins, lipids, or any other cellular components, such as mtDNA.

Suitably, oxidative stress may be caused by exposure to UVR and/or exposure to a pollutant (such as urban dust). Suitably exposure to UVR may be chronic or acute.

Exposure to UVR and/or pollution (such as urban dust) is known to increase oxidative stress. However, prior to the present disclosure, it was not previously known that the region of mtDNA located between nucleotides from 4412 to 7069, more suitably located between nucleotides from 4512 to 6969, is particularly susceptible to damage by exposure to UVR. This finding has allowed the inventors of the present disclosure to provide methods of determining and/or monitoring mtDNA caused by exposure to UVR and/or pollutants.

The term “UVR” as used herein refers to ultraviolet radiation that can be divided into three bands depending on wavelength: UVA, UVB, and UVC. UVA radiation is present in the sunlight reaching the earth's surface and has a wavelength of 320 to 400 nm. UVB radiation is present in the sunlight reaching the earth's surface and has a wavelength of 290 to 320 nm. Exposure to UVA immediately causes free iron to act as a catalyst in the production of ROS. As free iron concentrations are especially high inside the mitochondrial matrix, the mitochondria are highly susceptible to oxidative stress caused by UVA exposure, which can result in mtDNA damage. Oxidative stress caused by ROS, the production of which is catalysed by free iron, may be referred to as “iron induced oxidative stress”. Accordingly, in a suitable embodiment, oxidative stress may be iron induced oxidative stress.

The term “pollutant” as used herein refers to chemicals present in the environment (example in the air) that can cause mtDNA damage. Merely by way of example, the pollutants include urban dust, polycyclic aromatic hydrocarbons (PAH), oxides, particulate matter, ozone, and cigarette smoke. “Urban dust” also known as “smog” comprises particles and inorganic fibers which may include heavy metals, and toxic or carcinogenic organic compounds such as polycyclic aromatic hydrocarbon compounds, furans, aldehydes, which can even also be associated with pathogenic microorganisms. Such particles have sizes ranging from less than 1 μm up to 500 μm. The smaller these particles, the more their toxicity is increased, due to their ability to penetrate deeper into the epidermis.

The term “levels”, as used herein, refers to any measure of abundance and/or proportion of mtDNA damage in cell population, or sample of a cell population wherein the sample is representative of the cell population. It will be appreciated that a sample may be a subpopulation of the cell population. Suitable cell populations and/or samples of cell populations are described elsewhere in the present specification.

The method of determining the level of mtDNA damage in a cell population comprises the step of quantifying the total amount of mtDNA in a sample of the cell population.

The phrase “quantifying the total amount of mtDNA in a sample of the cell population” refers to determining the amount of healthy and damaged (for example by oxidative stress) mtDNA molecules in the sample. Methods of determining the total amount of mtDNA are well known in the art. Suitably, the step of quantifying the total amount of mtDNA in a sample may involve amplifying a region of mtDNA that can be found in both healthy and damaged mtDNA molecules. Such a region may be referred to as a “damage resistant mtDNA region”. Suitably, the damage resistant mtDNA region will not be substantially damaged by the same or similar levels of oxidative stress (such as oxidative stress caused by exposure to UVR and/or a pollutant) that would result in damaging of the regions of mtDNA described herein.

Suitably, the step of quantifying the total amount of DNA may comprise amplifying a damage resistant mtDNA region. Suitably, the damage resistant mtDNA region may be a fragment consisting of 100 bases or less. Suitably, the damage resistant mtDNA region may be a fragment consisting of 83 bases or less. More suitably, the damage resistant mtDNA region may be a fragment consisting of some or all of the bases located between nucleotides from 16042 to 16124. Most suitably, the damage resistant mtDNA region may be a fragment consisting of 83 or less bases located between nucleotides from 16042 to 16124. Such a fragment, may be amplified using a primer set having the nucleic acid sequences as shown in SEQ ID NO: 8 and 9.

Suitably the step of quantifying the total amount of mtDNA by amplifying the damage resistant mtDNA region may involve the use of a quantitative amplification method. Quantitative amplification methods are well known in the art. Such methods (e.g., quantitative PCR (qPCR) or quantitative linear amplification) involve amplification of a nucleic acid template (for example a damage resistant region of mtDNA), directly or indirectly (e.g., determining a Ct value) determining the amount of amplified DNA, and then calculating the amount of initial template based on the number of cycles of the amplification. Amplification of a DNA locus using reactions is well known (see U.S. Pat. Nos. 4,683,195 and 4,683,202; PCR Protocols: A Guide To Methods And Applications (Innis et al., eds, 1990)). Typically, PCR is used to amplify DNA templates. However, alternative methods of amplification have been described and can also be employed. Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34 (1): 106-10, 112-5 (2003); Deiman B, et al., Mol Biotechnol. 20 (2): 163-79 (2002). Amplifications can be monitored in “real time.”

Suitably amplification is by qPCR. Merely by way of example, the qPCR may be performed using a probe such as Lo-Rox SensiFASTTSYBR kit (Bioline). The total amount of mtDNA may be expressed, for example, in terms of weight and/or number of mtDNA molecules.

The method of determining the level of mtDNA damage in a cell population further comprises quantifying the amount of a mtDNA fragment that the inventors have found to be especially susceptible to damage by oxidative stress, such as oxidative stress caused by UVR exposure, and/or a pollutant.

The term “fragment” as used herein refers to an area of continuous, undamaged mtDNA. In the context of the present invention, the area (i.e. the fragment) comprises at least about 200 bases from a region of mtDNA located between nucleotides from 4412 to 7069. By “undamaged” it is meant that the mtDNA area defining the fragment does not contain any double and/or single stranded breaks. A single stranded break is a discontinuity in one strand of the double stranded DNA. Such a discontinuity may be as a result of breakage in at least one bond involved in forming the single strand. Such a breakage may be accompanied by a loss of one or more nucleotides in the one strand. A double stranded break is a discontinuity in both of the strands of the double stranded DNA. Such a discontinuity may be as a result of breakage in at least one bond involved in forming each of the single strands of the double stranded DNA.

The fragment comprises at least about 200 bases from a region of mtDNA located between nucleotides from 4412 to 7069. Thus, the method comprises quantifying the amount of a mtDNA fragment comprising at least about 200 bases from a region of mtDNA located between nucleotides from 4412 to 7069 in the sample of the cell population.

Methods for quantifying the total amount of DNA by amplifying a damage resistant mtDNA region as described herein above, apply equally to quantifying the amount of the mtDNA fragment comprising at least about 200 bases from a region of mtDNA located between nucleotides from 4412 to 7069. Accordingly, the fragment may be amplified using a quantification method such qPCR. Merely by way of example, the qPCR may be performed using a probe such as Lo-Rox SensiFAST™ SYBR kit (Bioline). The amount of damaged mtDNA may be expressed, for example, in terms of weight and/or number of mtDNA molecules.

Suitably the fragment may comprise at least about 200 bases, at least about 500 bases, at least about 650 bases, at least about 1000 bases, at least about 1200, at least about 1600, at least about 2000, or at least about 2400 bases from a region of mtDNA located between nucleotides from 4412 to 7069.

More suitably, the fragment may comprise at least about 200 bases, at least about 500 bases, at least about 650 bases, at least about 1000 bases, at least about 1200, at least about 1600, at least about 2000, or at least about 2400 bases from a region of mtDNA located between nucleotides from 4512 to 6969.

As used herein, the term “about” means within +10% of the indicated value. Thus, merely by way of example, a fragment that comprises about 1000 bases, is a fragment that comprises from 900 to 1100 bases.

Suitably, the fragment may comprise or consist of about 200 bases (for example 218 bases) located between nucleotides from 6067 to 6284. For example, such a fragment may have a sequence as shown in SEQ ID NO: 16, or a variant thereof.

Suitably, the fragment may comprise or consist of about 500 bases (for example 495 bases) located between nucleotides from 6067 to 6561. For example, such a fragment may have a sequence as shown in SEQ ID NO: 17, or a variant thereof.

Suitably, the fragment may comprise or consist of about 650 bases (for example 648 bases) located between nucleotides from 4619 to 5266. For example, such a fragment may have a sequence as shown in SEQ ID NO: 18, or a variant thereof.

Suitably, the fragment may comprise or consist of 1233 bases located between nucleotides from 4512 to 5744, or may comprise or consist of 1229 bases located between nucleotides from 5741 to 6969. For example, such fragments may have a sequence as shown in SEQ ID NO: 2 or 3, or a variant thereof.

Suitably, the fragment may comprise or consist of 2458 bases. Such a fragment may have a sequence as shown in SEQ ID NO: 1, or variant thereof. Suitably the 2458 bases fragment may be amplified using a primer set having amino acid sequences as shown in SEQ ID NO: 4 and 7.

As touched upon elsewhere in the present disclosure, the inventors found that when determining the levels of mtDNA damage using fragments that comprise 650 or less, it may be desirable to determine the levels 24 hours exposure to the mtDNA damaging factor, such as UVR and/or a pollutant (such as urban dust).

The variants of the sequences disclosed herein may arise as a result of a mutation being present within said sequences. The sequence of human mtDNA and mtDNA mutations are well known in the art. The sequence of human mtDNA and examples of known mtDNA mutations can be obtained from the MitoMap database available from https://www.mitomap.org/MITOMAP (version r105-28 Jan. 2022).

As mentioned elsewhere in the present disclosure, the present inventors, in addition to finding that the region of mtDNA located between nucleotides from 4512 to 6969 is susceptible to oxidative stress damage (for example caused by exposure to UVR and/or pollutants), also found that different parts of this fragment have different susceptibly to UVR induced damage, depending on whether the exposure to UVR and/or pollution is chronic or acute.

The term “chronic” as used herein refers to a low dose but prolonged exposure to an agent that causes mtDNA damage. It will be appreciated that in the context of the present discourse, such an agent may be UVR and/or pollution (for example urban dust).

The term “chronic UVR exposure” in the context of the present disclosure refers to a low dose of UVR exposure sustained over a prolonged time period, for example days, weeks, months or years. A low dose of UVR exposure is for example, 100 joules per square meter (J/m2) of erythemally weighted UVR, or 1 standard erythemal dose (1 SED). Suitably, in the context of the present disclosure chronic UVR exposure refers to exposure at a dose of 1 SED per day for a period of 3 days or more. Suitably the dose of 1 SED per day may be delivered in about 1 minute. 1 SED is equivalent to approximately 10 mins of Mediterranean sun at noon in midsummer. In vivo, such a low dose of UVR would not be generally expected to burn the cell population exposed (such as an exposed to the sun are of the skin) and therefore the effects of chronic UVR exposure may not be evident soon after (within minutes or hours) from exposure. However, over time, chronic exposure to UVR rays can cause premature aging of the skin and signs of sun damage such as wrinkles, leathery skin, liver spots, actinic keratosis, and solar elastosis.

The term “chronic pollution exposure” in the context of the present disclosure refers to a low dose of exposure to pollution (such as urban dust) sustained over a prolonged time period, for example days, weeks, months, or years. A low dose of pollution is for example about 25 ug/ml or less, about 20 ug/ml or less, about 15 ug/ml or less, about 10 ug/ml or less, or about 5 ug/ml or less. In vivo, such a low dose of pollution would not be generally expected to cause visible changes to the skin immediately or shortly upon exposure. However, over time, chronic exposure to pollution can cause, for example, premature aging of the skin, wrinkles, acne, and/or dryness.

Suitably, the exposure to UVR and/or pollution (for example urban dust) may be chronic.

Suitably, when exposure to UVR is chronic, the fragment may be from a region of mtDNA located between nucleotides from 4512 to 5744.

Suitably, the fragment from a region of mtDNA located between nucleotides from 4512 to 5744 may comprise at least about 200 bases, at least about 500 bases, at least about 650 bases, at least about 1000 bases, or at least about 1200 bases.

Suitably, the fragment may comprise or consist of about 650 bases (for example 648 bases) located between nucleotides from 4619 to 5266. For example, such a fragment may have a sequence as shown in SEQ ID NO: 18, or a variant thereof.

Suitably, the fragment may comprise or consist of 1233 bases. Such a fragment may have a sequence as shown in SEQ ID NO: 2, or a variant thereof. Suitably, such a fragment may be amplified by the primer set having nucleic acid sequences according to SEQ ID NO: 4 and 5.

Suitably, when exposure to pollution (such as urban dust) is chronic, the fragment may be from a region of mtDNA located between nucleotides from 5741 to 6969.

Suitably, the fragment from a region of mtDNA located between nucleotides from 5741 to 6969 may comprise at least about 200 bases, at least about 500 bases, at least about 650 bases, or at least about 1000 bases.

Suitably, the fragment may comprise or consist of about 200 bases (for example 218 bases) located between nucleotides from 6067 to 6284. For example, such a fragment may have a sequence as shown in SEQ ID NO: 16, or a variant thereof.

Suitably, the fragment may comprise or consist of about 500 bases (for example 495 bases) located between nucleotides from 6067 to 6561. For example, such a fragment may have a sequence as shown in SEQ ID NO: 17, or a variant thereof.

Suitably, the fragment may comprise or consist of 1229 bases. Such a fragment may have a sequence as shown in SEQ ID NO: 3, or be a variant thereof. Suitably, such a fragment may be amplified by the primer set having nucleic acid sequences according to SEQ ID NO: 6 and 7.

The variants of the sequences disclosed herein may arise as a result of a mutation being present within said sequences. Examples of known mtDNA mutations can be obtained from the MitoMap database mentioned hereinabove.

Suitably, chronic exposure to UVR may cause more damage in the mtDNA fragment located between nucleotides from 4514 to 5744, whereas chronic exposure to pollution may cause more damage to the mtDNA fragment located between nucleotides from 5741 to 6969. The inventors believe that this difference may be caused by the different mechanisms that these effects (UVR and pollution) operate under. The same may apply to explain the different fragments preferentially damaged due to exposure to acute UVR and acute pollution as discussed below.

The term “acute” as used herein refers to a high dose exposure over a short time period to an agent that causes mtDNA damage. It will be appreciated that in the context of the present disclosure, such an agent may be UVR and/or pollution (for example urban dust).

The term “acute UVR exposure” in the context of the present disclosure, refers to a high dose of UVR exposure over a short time period (for example minutes or hours). Such a high dose of UVR exposure is for example, joules per square meter (J/m2) of erythemally weighted UVR, or 3 SEDs. Suitably, in the context of the present disclosure acute UVR exposure refers to exposure at a dose of 3 SEDs per day. Suitably the dose of 3 SED per day may be delivered in about 1 to 10 minutes, more suitably in about 3 minutes. This is equivalent to approximately 30 mins of Mediterranean sun at noon in midsummer. Acute UVR exposure may result in injury (such as sunburn) of the cell population (such as an exposed to the sun are of the skin). Acute UVR injury or sunburn may result in cell death in the upper skin layer or epidermis.

The term “acute pollution exposure” in the context of the present disclosure, refers to a high dose of exposure to pollution (such as urban dust) sustained over a short period of time, for example a day, hours, or minutes. A high dose of pollution is for example more than 25 ug/ml. A high dose of pollution may therefore be 30 ug/ml, 50 μg/ml, 75 ug/ml, 100 μg/ml, 150 ug/ml, 200 ug/ml or more. Acute exposure to pollution may cause visible changes to the skin immediately or shortly (for example within about 30 mins, 1 hr, 2 hrs, or 3 hrs) after exposure. Suitably, the exposure to UVR and/or pollution (for example urban dust) may be acute.

Suitably, when exposure to UVR is acute, the fragment may be from a region of mtDNA located between nucleotides from 5741 to 6969.

Suitably, the fragment from a region of mtDNA located between nucleotides from 5741 to 6969 may comprise at least about 200 bases, at least about 500 bases, at least about 650 bases, or at least about 1000 bases.

Suitably, the fragment may comprise or consist of about 200 bases (for example 218 bases) located between nucleotides from 6067 to 6284. For example, such a fragment may have a sequence as shown in SEQ ID NO: 16, or a variant thereof.

Suitably, the fragment may comprise or consist of about 500 bases (for example 495 bases) located between nucleotides from 6067 to 6561. For example, such a fragment may have a sequence as shown in SEQ ID NO: 17, or a variant thereof.

Suitably, the fragment may comprise or consist of 1229 bases. Such a fragment may have a sequence as shown in SEQ ID NO: 3, or be a variant thereof. Suitably, such a fragment may be amplified by the primer set having nucleic acid sequences according to SEQ ID NO: 6 and 7.

Suitably, when exposure to pollution (for example urban dust) is acute, the fragment may be from a region of mtDNA located between nucleotides from 4512 to 5744.

Suitably, the fragment from a region of mtDNA located between nucleotides from 4512 to 5744 may comprise at least about 200 bases, at least about 500 bases, at least about 650 bases, at least about 1000 bases, or at least about 1200 bases.

Suitably, the fragment may comprise or consist of about 650 bases (for example 648 bases) located between nucleotides from 4619 to 5284. For example, such a fragment may have a sequence as shown in SEQ ID NO: 18, or a variant thereof.

Suitably, the fragment may comprise or consist of 1233 bases. Such a fragment may have a sequence as shown in SEQ ID NO: 2, or a variant thereof. Suitably, such a fragment may be amplified by the primer set having nucleic acid sequences according to SEQ ID NO: 4 and 5.

The variant may arise as a result of a mutation being present within SEQ ID NO: 3. mtDNA mutations are well known in the art. Examples of known mtDNA mutations can be obtained from the MitoMap database as mentioned hereinabove.

When referring to the nucleotide positioning herein, these positions are based on the standard numbering of the human mtDNA sequence. The skilled person will have no difficulty in determining the precise positioning of these fragments, as the full sequence of human mtDNA is known. For avoidance of doubt, the human mtDNA sequence has a sequence as provided in NCBI Reference Sequence: NC_012920.1.

As used herein, the term “cell population” refers to a group of at least two cells (for example at least about 10, at least about 100, at least about 200, at least about 300, at least about 400, at least about 500, at least about 600, at least about 700, at least about 800, at least about 900, at least about 1000 cells, or more cells). The cells within a cell population may be expected to have similar or substantially the same levels of mtDNA damage. By “similar levels” as used herein, it is meant that the levels of mtDNA damage within the cells of the cell population differ by less than 15%, less than 10%, or less than 5%. Cells may be expected to have similar or substantially the same levels of mtDNA damage due to having been exposed to approximately the same levels to UVR and/or pollutant. Merely by way of example cells located on the same body part (for example face) may be expected to have similar or substantially the same levels of mtDNA damage, due to being exposed to approximately the same levels to UVR and/or pollutant.

Suitably, the cell population may be a skin cell population. A skin cell population may be in an in vivo setting (i.e. growing on a subject, such as a mammal, for example a human, a cat, a dog, or a farm animal). Suitably the skin cell population that is in an in vivo setting may comprise of epidermal and/or dermal cells.

In vivo, different skin sites may have cells with different mtDNA damage levels. This may be due to different amounts of exposure to UVR and/or pollutants. Accordingly, a skin cell population may be the skin cells located on a specific (single) body part (for example, face, scalp. shoulders, neck, cheek, back, arm, inner arm, thigh, inner thigh, etc.). Suitably, a skin cell population may be the cells located within the same square centimetre or square inch of skin. For avoidance of doubt, it shall be appreciated that the skilled person would be capable of determining whether skin cells located on a specific body part would have similar levels of mtDNA damage.

Alternatively, a skin cell population may be in an in vitro setting (i.e. cultured). A skin cell population that is cultured may suitably comprise or consist of fibroblasts. It will be appreciated that a single cell population of fibroblasts, may or may not be cultured in the same cell culture flask, plate or well. For example a single fibroblast cell population may be cultured in two, three, four, five, six or more cell culture flasks, plates or wells.

Suitably, the cells (such as fibroblasts) in an in vitro setting may be located in an in vitro skin model. Such an in vitro skin model may comprise other cell types typically associated with fibroblasts in vivo.

Suitably, cell population may be a pool of many cell populations. Such a pool may be for example a mixture of skin cell populations from different individuals and/or from different body parts.

The term “sample of a cell population” as used herein refers to a portion of the cells in the cell population. Suitably the sample is a sample of a skin cell population. When the skin cell population is in an in vivo setting, such a sample may be obtained by swabbing the skin to collect skin cells. Suitably, the collected skin cells may be epidermal cells. An exemplary method of swabbing the skin to collect cells is provided in the Examples section of the present disclosure. Merely by way of example, the area of skin from which a sample is to be taken may be cleaned with a 2% chlorhexidine in 70% isopropyl alcohol skin wipe to clean and left to air dry prior to obtaining the sample. The cleaned area may be swabbed 30 times up and down to obtain the sample.

It will be appreciated that by measuring the levels of mtDNA damage in a sample, one is able to determine the levels of mtDNA damage in the sample population from which the sample is derived from, and optionally at the time in which the sample was obtained from the cell population.

In order to determine the levels of mtDNA damage, the method comprises the step of comparing the amount of the fragment comprising at least about 200 bases from a region of mtDNA located between nucleotides from 4412 to 7069 in the sample of the cell population to the amount of total mtDNA.

The term “comparing” as used herein refers to any suitable method of assessing, calculating, evaluating or processing of data relating that allows one to determine relative or absolute amount of the fragment as compared to the total level of mtDNA in the sample. The comparison may be carried out manually or be computer assisted. The levels of mtDNA may be suitably represented as a fraction or percentage.

Suitably, the method of determining the levels of mtDNA damage as described herein may comprise the step of extracting mtDNA from the sample prior to step a) of the method. Methods of extracting mtDNA are well known in the art. MtDNA may be extracted using BuccalPrep Plus kit (IsoHelix, UK) or QIAampDNA Mini Kit (QIAGEN, Europe) according to manufacturers' instructions.

The steps of the method of determining the levels of mtDNA damage need not to be carried in order of step a), followed by step b), then followed by step c). It shall be apparent to a person skilled in the art, that step b) may be carried out prior to step a). It will also be apparent to the person skilled in the art that steps a) and b) may be carried sequentially or simultaneously.

In a further aspect, the present invention provides a method of determining the ability of a test agent to prevent or repair mtDNA damage in a cell population, the method comprising:
a) determining the level of mtDNA damage in a first sample of the cell population, wherein the level of mtDNA damage is determined by a method of the first aspect as described herein;
b) providing the test agent to the cell population;
c) determining the level of mtDNA damage in a second sample of the cell population, wherein the level of mtDNA damage is determined by a method of the first aspect as described herein; and
d) comparing the level of mtDNA damage determined in step c) to the levels of mtDNA damage determined in step a); wherein
   i) no change between the level of mtDNA damage determined in step c) as compared to step a) is indicative of the test agent having the ability to prevent mtDNA damage; or
   ii) a decrease between the level of mtDNA damage determined in step c) as compared to step a) is indicative of the test agent having the ability to repair mtDNA damage.

The term “test agent” refers to an agent that is evaluated to determine whether it has the ability to alter the levels of mtDNA damage, for example to prevent or repair mtDNA damage. The test agent may be a cosmetic or a therapeutic. The term “cosmetic” as used herein refers to agents intended to be rubbed, poured, sprinkled, or sprayed on, introduced into, or otherwise applied to the human body or any part thereof (for example the skin) for cleansing, beautifying, promoting attractiveness, and/or altering the appearance. Accordingly, the test agent may be, by way of example, a cleanser, serum, extract, toner, cream, gel, oil, etc. In another example the cosmetic test agent may be a supplement intended for internal administration (for example oral administration).

The term “therapeutic” as used herein refers to agents intended to reduce mtDNA damage treat or prevent a disease associated with mtDNA damage. An example associated with mtDNA damage is skin cancer.

The term “providing” as used herein means exposing the cell population to the test agent. The cell population may be exposed to the test agent by contacting or placing the test agent over the cell population. In some examples, placing the test agent over the cell population may create a physical barrier reducing the cells exposure to a mtDNA damaging factor, such as UVR and/or a pollutant. It will be appreciated that when the cell population is in vitro, the cell population may be exposed to the test agent, by administering the test agent, or a precursor of the test agent to the subject. The precursor may be metabolised to the test agent upon administration to the subject. Merely by way of example, the test agent may be administered orally, intravenously, or subcutaneously.

The amount, frequency and duration that the cells are exposed to the test agent may differ depending on the formulation, clinical indication, age, and route of administration. Suitably, when the cell population is skin, the route of administration may be topical.

It will be appreciated that not all cells of the cell population will necessarily come into direct contact with the test agent (for example when the cell population is a skin cell population in an in vivo setting) for the test agent to be applied. Suitably, only the outer layer(s) may come into contact with the test agent. However, by providing the test agent to the outer layer(s), layer(s) beneath the outer layer(s) may be protected from exposure to UVR and/or a pollutant. Such protection may in turn result in mtDNA damage being prevented or repaired in the whole cell population.

Suitably, the agent is applied in a “therapeutically effective amount”. This is an amount, that when applied to the cell population, is sufficient to reduce mtDNA damage. The term “reduce” mtDNA damage encompasses the terms prevent and/or repair mtDNA damage. The term “prevent,” as used herein, refers to a prophylactic action that stops an increase or reduces the rate at which mtDNA damage is increased upon exposure to UVR and/or a pollutant as compared to a suitable control. A suitable control may be for example a population of cells to which the test agent has not been applied. The reduction may be for example be by at least about 5%, at least about 10%, at least about 20%, at least about 30%, at least about 40%, at least about 50%, at least about 60%, at least about 70%, at least about 80%, at least about 90% or more.

The term “repair” as used herein refers to a process by which damage to mtDNA is reduced or eliminated altogether after mtDNA damage due to exposure to UVR and/or a pollutant had already occurred. The reduction may be, for example, by at least about 5%, at least about 10%, at least about 20%, at least about 30%, at least about 40%, at least about 50%, at least about 60%, at least about 70%, at least about 80%, at least about 90% or more. For the purpose of determining if a test agent repairs mtDNA, it may be preferable to determine mtDNA damage using a fragment of mtDNA that comprises at least 1000 bases from a region of mtDNA located between nucleotides from 4412 to 7069 in the sample of the cell population.

In the context of a method for determining the ability of a test agent to prevent mtDNA damage, the method may comprise the step of exposing the cell population to UVR and/or a pollutant after the test agent has been applied to the cell population.

In a further aspect, the present invention provides a method of monitoring progression of mtDNA damage in a cell population, the method comprising:
a) determining the level of mtDNA damage in a first sample of the cell population, wherein the level of mtDNA damage is determined by a method of the first aspect as described herein;
b) determining the level of mtDNA damage in a second sample of the cell population according to the invention, wherein the level of mtDNA damage is determined by a method of the first aspect as described herein, and wherein the second sample has been obtained from the cell population at a later time point than the first sample; and
c) comparing the amount of mtDNA damage determined in step b) as compared to step a); wherein an increase in mtDNA damage in step b) as compared to step a) is indicative of progression of mtDNA damage.

The term “monitoring” as used herein refers to determining mtDNA damage levels in a cell population over time, without providing to the cell population any test agent aimed at reducing (i.e. repairing or preventing mtDNA damage). Accordingly, the second sample of the cell population is obtained from the cell population at a later time point. Such a later time point, may be for example about 1 hour, about 12 hours, about 1 day, about 3 days, about 1 week, about 1 months or about 1 year later. During the time between obtaining the first and second sample from the cell population, the cell population may be exposed to UVR and/or a pollutant.

It can be said that mtDNA damage has progressed when the levels of mtDNA damage obtained in step b) as compared to step a) have increased. By “increased” it is meant that there are statistically significantly higher levels of mtDNA damage in step b) as compared to step a). An increase, may be of at least 10% or more (for example of about 20%, about 30%, about 40%, about 50%, about 60%, about 70%, about 80%, about 90%, about 100% or more).

In the event that the levels of mtDNA in step b) are decreased as compared to step a) it can be said that mtDNA damage has been reduced. By “reduced” it is meant that there are statistically significantly lower levels of mtDNA damage in step b) as compared to step a). An reduction, may be of at least 10% or more (for example of about 20%, about 30%, about 40%, about 50%, about 60%, about 70%, about 80%, about 90%, or more).

In the event that there is no change in levels of mtDNA damage in step c) as compared to step a), it can be said that mtDNA has not progressed.

### Kits and Uses

In a further aspect, the present invention provides a kit for determining the level of mitochondrial DNA (mtDNA) damage in a cell population comprising a primer set for amplifying a mtDNA fragment comprising at least 200 bases from a region of mtDNA located between nucleotides from 4412 to 7069.

Suitably, the primer set may comprise the nucleic acid sequences selected from the group consisting of 1) SEQ ID NO: 4 and SEQ ID NO: 7; (2) SEQ ID NO: 4 and SEQ ID NO: 5; (3) SEQ ID NO: 6 and SEQ ID NO: 7; (4) SEQ ID NO: 10 and SEQ ID NO: 11; (5) SEQ ID NO: 12 and SEQ ID NO: 13; and (5) SEQ ID NO: 14 and SEQ ID NO: 15.

Suitably, the kit may further comprise a primer set for amplifying a damage resistant region of mtDNA. Such a damage resistant region may be as defined elsewhere in the present disclosure. Suitably such a primer set may comprise nucleic acid sequences according to SEQ ID NO: 8 and SEQ ID NO: 9.

As used herein the term “primer set” refers to at least two primers, wherein at least one of the two primers is a forward primer and at least one of the two primers is a reverse primer.

Suitably, the kit may comprise a positive control substance and a negative control substance.

Suitably, the kit may comprise a labelled probe and/or DNA polymerase. Merely by way of example the labelled probe may be Lo-Rox SensiFAST™ probe (Bioline).

Suitably, the kit may comprise DNA standards for obtaining a DNA standard curve.

In a further aspect, the present invention provides use of a mtDNA fragment comprising at least 200 bases from a region of mtDNA located between nucleotides from 4412 to 7069 for determining or monitoring the level of mtDNA damage. Suitably, the fragment may be located between nucleotides from 4512 to 6969.

### EXAMPLES

### Example 1-UV Damage MtDNA Damage

### 1 Materials and Methods

### 1.1 Skin Swab and DNA Extraction

Skin swabs were taken from the left cheek, right cheek, behind the ear and the inner arm. A large area of skin was wiped thoroughly with a 2% chlorhexidine in 70% isopropyl alcohol skin wipe to clean and left to air dry. The cleaned area was swabbed 30 times up and down. Swabs were cut using 70% ethanol cleaned scissors into a 1.5 mL microfuge tube, and stored until subsequent DNA extraction using BuccalPrep Plus kit (IsoHelix, UK). QIAampDNA Mini Kit (QIAGEN, Europe) following manufacturers' instructions may also be used.

### 1.2 Amplification of 83 bp Region

A small, 83 bp region of mtDNA was amplified to normalise mtDNA copy number. PCR was performed in 20 μl reaction containing: 2 μl of each DNA sample, 400 UM of each primer, 1×Lo-Rox SensiFAST™ SYBR kit (Bioline) and made up to the correct volume using UVR sterile, high grade PCR water. Primer sequences are in Table 1 (previously described in Hanna et al. Mitochondrion. 2019 May; 46:172-178). Cycling condition were as follows: 2 min initial denaturation stage at 94° C. followed by 35 cycles of 15 s denaturation at 94° C., 45 s annealing at 60° C. and 45 s extension at 72° C., and a 2 min final extension at 72° C.

TABLE 1

| Primer | Sequence | SEQ ID NO: |
|---|---|---|
| Forward | 5′- GATTTGGGTACCACCCAAGTATTG -3′ | 8 |
| IS1 | | |
| Reverse | 5′- AATATTCATGGTGGCTGGCAGTA -3′ | 9 |
| IS2 | | |

### 1.3 Amplification of Combined Target Region

A larger, 2458 bp region of mtDNA was amplified to detect mtDNA damage. PCR was performed in 20 μl reaction containing: 2 μl of each DNA sample, 200 UM of each primer, 1×Lo-Rox SensiFAST™ SYBR kit (Bioline) and made up to the correct volume using UVR sterile, high grade PCR water. Primer sequences are in Table 2. Cycling condition were as follows: 10 min initial denaturation stage at 95° C. followed by 35 cycles of 15 s denaturation at 95° C., 20 s annealing at 60° C. and 90 s extension at 72° C., and a 7 min final extension at 72° C.

TABLE 2

| Primer | Binding region | Sequence | SEQ ID NO: |
|---|---|---|---|
| Forward 6F | 4512-4532 | 5′- GCAGGCACACTCATCACAGCG-3′ | 4 |
| Reverse 7R | 6950-6969 | 5′- TGCCAGTCAGGCCACCTACG -3′ | 7 |

### 1.4 Amplification of Two Target Regions

Two regions within the 2458 bp region of mtDNA were amplified, of sizes 1233 bp (primer set 6) and 1229 bp (primer set 7) to detect mtDNA damage. PCR was performed in 20 μl reaction containing: 2 μl of each DNA sample, 200 UM of each primer, 1×Lo-Rox SensiFAST™ SYBR kit (Bioline) and made up to the correct volume using UVR sterile, high grade PCR water. Primer sequences are shown below (Table 3). Cycling condition were as follows: 10 min initial denaturation stage at 95° C. followed by 35 cycles of 15 s denaturation at 95° C., 15 s annealing at 60° C. and 55 s extension at 72° C., and a 7 min final extension at 72° C.

TABLE 3

| | | | SEQ ID |
|---|---|---|---|
| Primer | Binding region | Sequence | NO: |
| Forward 6F | 4512-4532 | 5′- GCAGGCACACTCATCACAGCG-3′4 | 4 |
| Reverse 6R | 5723-5744 | 5′- CGGCGGCGGGAGAAGTAGATTG -3′ | 5 |
| Forward 7F | 5741-5760 | 5′- GCCGGGAAAAAAGGCGGGAG -3′ | 6 |
| Reverse 7R | 6950-6969 | 5′- TGCCAGTCAGGCCACCTACG -3′ | 7 |

### 1.5 Amplification of Smaller Target Regions

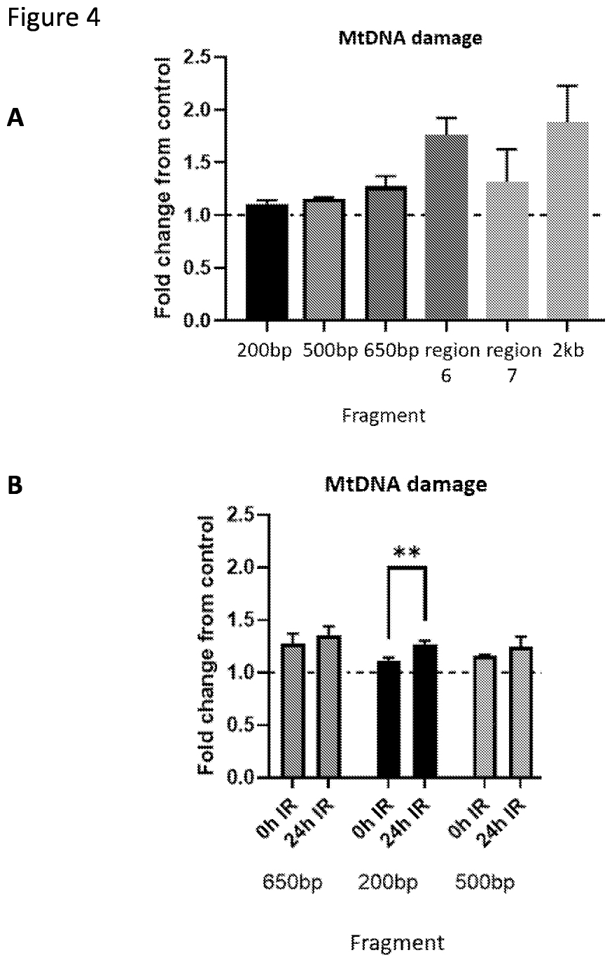

The inventors determined mtDNA damage in smaller fragments of 218, 500 and 650 bps. As shown in FIG. 4 such smaller fragments also enabled the inventors to reliably detect mtDNA damage caused by UVR exposure. The table (Table 4) below provides primers used to amplify these smaller fragments.

TABLE 4

| | Binding | | SEQ ID |
|---|---|---|---|
| Primer | region | Sequence | NO: |
| Forward | 6067-6088 | ACGTTATCGTCACAGCCCATGC | 10 |
| 218 bp | | | |
| Reverse | 6284-6263 | TGTTCAACCTGTTCCTGCTCCG | 11 |
| 218 bp | | | |
| Forward | 6067-6088 | ACGTTATCGTCACAGCCCATGC | 12 |
| 495 bp | | | |
| Reverse | 6561-6540 | AGAAGGTGGTGTTGAGGTTGCG | 13 |
| 495 bp | | | |
| Forward | 4619-4640 | TCGTTCCACAGAAGCTGCCATC | 14 |
| 648 bp | | | |
| Reverse | 5266-5245 | ATGGCCCATTTGGGCAAAAAGC | 15 |
| 648 bp | | | |

### 2 Results and Discussion

Primers were designed to determine mtDNA damage in regions of approximately 1 kb in size to cover the entire mtDNA genome. Dermal fibroblast cell lines were used to determine the level of damage in each region in response to chronic solar light (3 doses of 100 mJ/m2 delivered at a rate of 1 dose per day) and an acute dose (300 mJ/m2 delivered in one day). mtDNA damage was determined immediately after UVR exposure and at 24 hours after exposure. Region 6 was found to have a significant reduction in damage over 24 hours in response to chronic UVR exposure. Region 7 was found to have a significant increase in damage in response to an acute dose. These regions (6 and/or 7) had more substantial changes in mtDNA damage compared to any of the other 14 tested regions. A combination of both these regions was then tested, and there was an additive effect of the damage due to acute and/or chronic UVR exposure when both regions were amplified together as an approximately 2 kb amplicon.

Fragments of 218 (shown as 200 bp in FIG. 4), 500 and 650 bases were also found to have increased mtDNA damage immediately upon completion of chronic UVR exposure as compared to a control that was not exposed to UVR. In these shorter fragments mtDNA damage was even more evident 24 hours upon chronic UVR exposure completion.

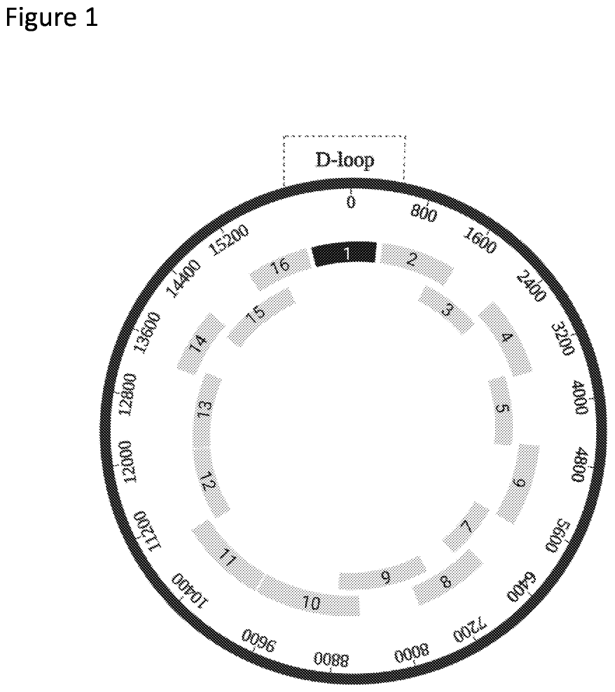

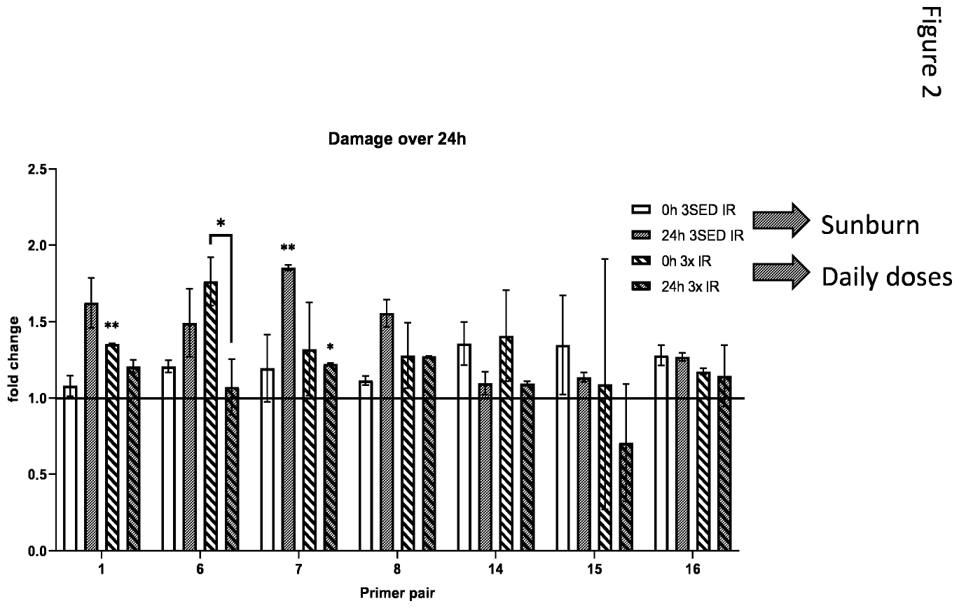

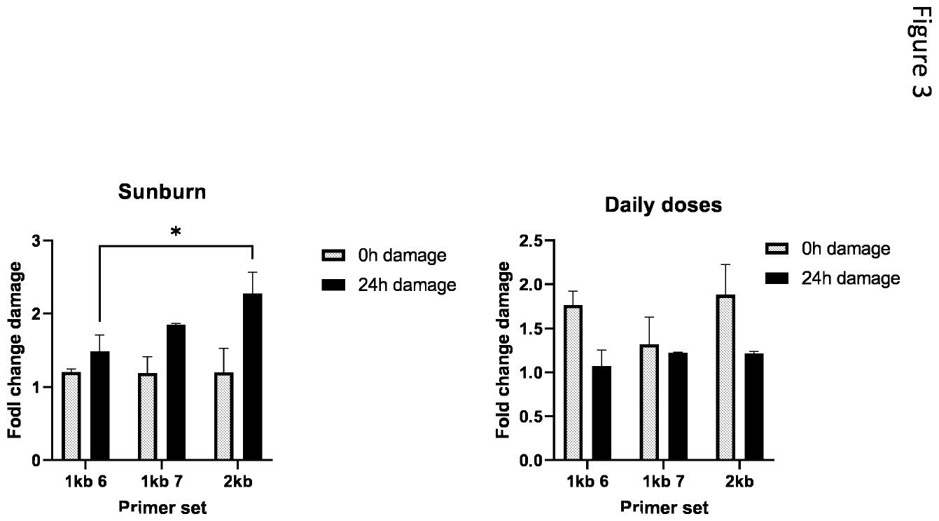

It shall be noted that in FIGS. 1 to 4, 200 bp refers to 218 bp, 500 bp refers to 495 bp, 650 refers to 648 bp. Furthermore, 1 kb 6 refers to region 6 which is in fact 1233 bp, 1 kb 7 refers to region 7 which is in fact 1229 bp, and 2 kb refers to combined regions 6 and 7 which are in fact 2458 bp.

### Example 2-Urban Dust MtDNA Damage

### Materials and Methods

### General Cell Culture

The HDFn cell line (Invitrogen, UK) was cultured in Dulbecco's modified Eagles medium (DMEM); 4.5 g/l-glucose containing L-glutamine, sodium pyruvate and sodium bicarbonate (Sigma-Aldrich UK). DMEM was supplemented with 10% foetal bovine serum (FBS) and 1% penicillin and streptomycin. At 80-90% confluency, cells were washed with PBS and detached using trypsin-EDTA solution (Sigma-Aldrich, UK). Trypsin-EDTA solution was neutralised with medium. Following trypsinisation, a small volume of cell suspension was added to an equal volume of 0.2% w/v trypan blue solution (Thermo Fisher, USA) and loaded onto a disposable countless cell counting chamber slide. This trypan blue exclusion assay measures cell viability, with the dye being able to permeate the membrane of dead cells and stain them blue, but not being able to permeate the membrane of viable cells. Total cell number and cell viability in a known volume were used to accurately calculate viable cell seeding densities for experiments.

For experiments, a cell suspension of 100,000 cells/ml was made up, and 2 ml cell suspension was added to each well of a 6-well plate, including irradiated control wells. Cells were incubated overnight to adhere.

### Urban Dust Preparation

Urban dust was used to mimic environmental pollution. Urban dust was made up at 1000× the required concentration in DMSO, to ensure that the amount of DMSO added to each well was equal. Urban dust was then diluted in media to the appropriate concentration and 2 ml was added to each well. Final concentrations were 0, 5, 10, 25, 50 and 100 ug/ml. Urban dust, in particular urban dust particulate matter” (PM) was purchased from the National Institute of Standards and Technology (NIST).

### Irradiation

A Newport solar simulator (MKS Instruments, Inc., USA) was used to irradiate positive control cells to the relevant standard erythemal dose (SED). Following overnight incubation, cells within the irradiation group were irradiated with 2.16 SED to mimic approximately 20 minutes in the Mediterranean sun. This allowed the comparison of damage caused by both UVR and urban dust. Media was removed and cells were washed twice PBS, before replacing with fresh PBS for irradiation. Cells were in PBS for no longer than 10 minutes to avoid cell stress. An ILT-1400 (International Light Technologies, USA) handheld radiometer/photometer was used to measure radiant energy. Calculations were performed previously in house to determine relevant dose timings. Following irradiation, PBS was replaced with 2 ml warmed phenol red-free DMEM and the cells were returned to the incubator for before harvesting.

### DNA Harvesting

Cells were harvested at both 8 hours and 24 hours following treatment with urban dust or UVR. Cells were washed with PBS and detached using trypsin-EDTA solution (Sigma-Aldrich UK). Trypsin-EDTA solution was neutralised with medium. The cell suspension was transferred to 1.5 ml Eppendorf tubes and centrifuged for 5 minutes at 1400 rpm. The supernatant was removed, paying attention to not disturb the cell pellet, and cell pellets were washed with PBS. This was followed by a second centrifuge at 1400 rpm for 5 minutes, before aspirating the supernatant. DNA was extracted using the QIAamp DNA Mini Kit (QIAGEN), following the manufacturers' instructions, and DNA was eluted in 60 μl TE buffer. Other extraction kits such BuccalPrep Plus kit (IsoHelix, UK) according to manufacturer's instructions may be used.

### 83 Amplicon (Housekeeping Fragment)

An 83 bp region known as the “housekeeping region” of the mitochondrial genome was amplified to quantify total mtDNA within each sample. Primers bind to and amplify 16042-16234 bp, a region within the mitochondrial D-loop. The relative mtDNA copy number was determined by the number of cycles required for the level of amplified product the reach the threshold, known as the cycle threshold (Ct) value. Fewer number of cycles indicates a higher concentration of mtDNA within the sample as there is more DNA available to be amplified.83 bp regions of the mitochondrial genome were amplified in a 20 μl reaction. SYBR Green binds to double stranded DNA and fluorescence relative to a passive ROX reference dye. Each sample was assayed in triplicate, with a standard deviation threshold of ≤0.3 Ct. The threshold was set to automatic threshold. Mastermix composition, amplification settings and primer sequences are presented in Table 5, Table 6 and Table 1, respectively.

TABLE 5

| Mastermix composition | Volume (μl) | Final concentration |
|---|---|---|
| Forward primer (10 μM) | 0.8 | 0.4 uM |
| (Eurofins, Europe) | | |
| Reverse primer (10 μM) | 0.8 | 0.4 uM |
| (Eurofins, Europe) | | |
| SensiMix Low-ROX (2X) | 10 | 1X |
| (Scientific Laboratory | | |
| Supplies, UK) | | |
| PCR grade H₂O (Thermo | 6.4 | N/A |
| Fisher Scientific, USA) | | |
| Mastermix total | 18 | |
| DNA | 2 | |
| Reaction total | 20 | |

TABLE 6

| Stage | Temperature (° C.) | Time (minutes) | Cycles |
|---|---|---|---|
| Initial denaturation | 95 | 10:00 | 1 |
| Denaturation | 95 | 0:15 | 40 |
| Annealing | 60 | 0:15 | |
| Extension | 72 | 0:55 | |
| Final extension | 72 | 07:00 | |
| Melt curve analysis | | | |

### 1232 bp and 1229 bp Amplicons

1233 bp (primer set 6) and 1229 bp (primer set 7) regions of the mitochondrial genome were amplified to quantify damage in the form of strand breaks. Primers bind to and amplify regions 4512-5744 bp and 5741-6969 bp respectively (see Table 3). A high Ct value corresponds to a large amount of mtDNA damage within a sample, as there is less intact mtDNA available to be amplified which requires more cycles to reach the cycle threshold.

Regions were amplified in 20 μl reactions, and assays were run on the same plate as the 83 bp assay. As previously mentioned, SYBR Green binds to double stranded DNA and fluoresces relative to a passive ROX reference dye. Each sample was assayed in triplicate with a standard deviation threshold of ≤0.3 Ct. The threshold was set to automatic threshold. Mastermix composition and primer sequences are presented in Table 7 and Table 3, respectively. Amplification settings are presented in Table 6.

TABLE 7

| Mastermix composition | Volume (μl) | Final concentration |
|---|---|---|
| Forward primer (10 μM) | 0.5 | 0.25 uM |
| (Eurofins, Europe) | | |
| Reverse primer (10 μM) | 0.5 | 0.25 uM |
| (Eurofins, Europe) | | |
| SensiMix Low-ROX (2X) | 10 | 1X |
| (Scientific Laboratory | | |
| Supplies, UK) | | |
| PCR grade H₂O (Thermo | 7 | N/A |
| Fisher Scientific, USA) | | |
| Mastermix total | 18 | |
| DNA | 2 | |
| Reaction total | 20 | |

### Analysis

Data was manipulated in Microsoft Excel (USA) and analysed using GraphPad Prism Version 9. PCR data was analysed using ddCt methodology, with an increase in fold change corresponding to increased damage within the mitochondrial genome.

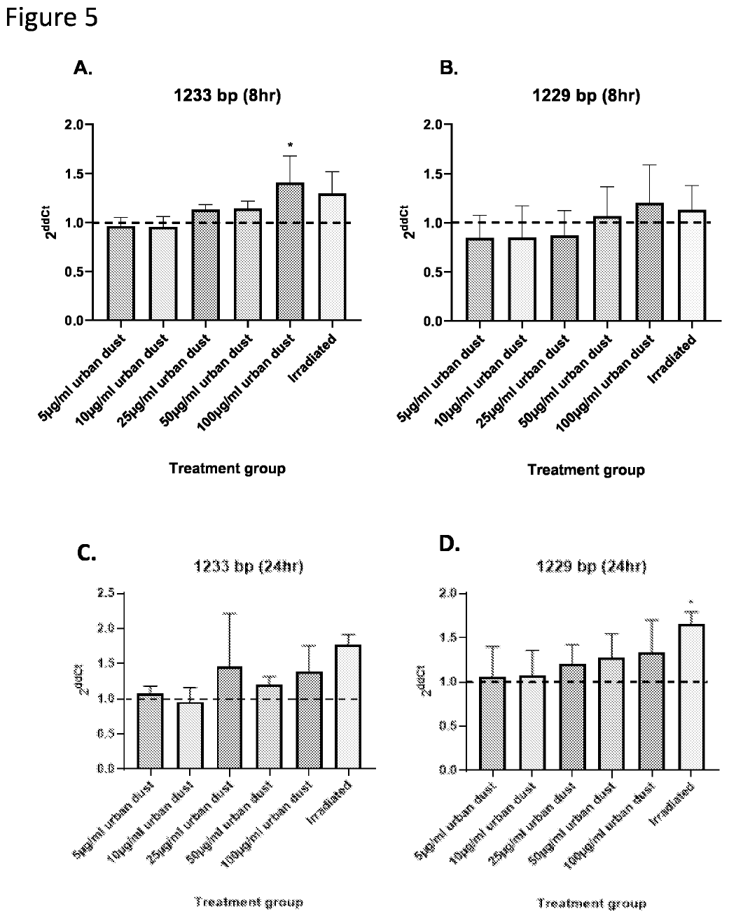

### Results (FIG. 5)

### 8 Hour Incubation

Following treatment with urban dust over a range of increasing concentrations for 8 hours, damage using the 1233 bp and 1229 bp strand break assays was assessed, respectively (region 6 and 7 respectively, FIG. 1).

When compared to the control, damage increased with increasing concentrations of urban dust to 1.411 at 100 ug/ml urban dust (p=0.02) with the 1233 bp assay (FIG. 5A). The irradiated treatment group was close to significant (p=0.11), with a value of 1.298.

Significance was not seen with the 1229 bp assay at 8 hours (FIG. 5B). Although a decrease in damage was observed with 10 and 25 ug/ml urban dust, an increase in damage was present with 50 and 100 μg/ml, with values of 1.069 and 1.204 respectively. The irradiated treatment group had a value of 1.133, lower than that with the 1233 bp assay.

Overall, a more consistent increase in damage was observed with the 1233 bp assay with increasing concentrations of urban dust, as well as a greater level of damage with both urban dust and UVR.

### 24 Hour Incubation

Following treatment with urban dust over a range of increasing concentrations for 24 hours, damage using the 1233 bp and 1229 bp strand break assays was assessed, respectively.

When compared to the control, damage was greater with 25, 50 and 100 ug/ml urban dust with the 1233 bp assay (FIG. 5C); however the increase in damage is not consistent with increasing concentrations of urban dust. The irradiated group showed the greatest level of damage with a value of 1.769 and was close to being significant (p=0.06).

An increase in damage was observed with increasing concentrations of urban dust with the 1229 bp assay (FIG. 5D); however, significance was not observed. The irradiated group showed the greatest level of damage with a value of 1.657, which was statistically significant (p=0.04). Damage induced by UVR was greater at 24 hours in comparison to 8 hours.

### Discussion of Results

In summary, following treatment for 8 hours, the 1233 bp assay (region 6 in FIG. 1 and Table 3) showed a more consistent increase in damage to the mitochondrial genome, with increasing concentration of urban dust resulting in increased mtDNA damage. In contrast, the 1299 bp assay (region 7, FIG. 1 and Table 3) showed a decrease in damage relative to the control for 10 and 25 ug/ml urban dust; however, an increase in damage was seen in groups treated with 50 and 100 ug/ml urban dust. The greatest level of damage was observed with 100 ug/ml urban dust with the 1233 bp assay, which was statistically significant (p=0.11). With both assays, the level of damage within the irradiated groups was greater than the damage initiated by 50 ug/ml urban dust, but less than that initiated by 100 ug/ml urban dust.

In comparison, a consistent increase in damage was not observed at 24 hours with the 1233 bp assay, unlike the 1299 bp assay which showed a consistent increase in damage with increasing concentrations of urban dust. As well as this, the irradiated groups for both assays at 24 hours showed the greatest level of damage in comparison to all urban dust treatment groups.

The results suggest that the 1233 bp assay is more efficient at detecting short term damage, as the data suggest that the damage begins to repair at 24 hours with this assay. On the other hand, the 1229 bp assay appears to detect damage more effectively at 24 hours, in comparison to 8 hours. As well as this, the data suggests that both assays detect a greater level of damage following irradiation at 24 hours, in comparison to at 8 hours.

In conclusion, the results suggest that the assays designed and described herein can be used to detect damage caused by both UVR and environmental pollution. Following further work and optimisation, the assays therefore can detect damage caused by these stressors in samples obtained using a skin swab to harvest skin cells from the epidermal layer. This data therefore supports that damage to the mitochondrial genome can be detected following exposure to environmental pollution as well as UVR.

### REFERENCES

1. Venus M, Waterman J, McNab I. Basic physiology of the skin. Surgery (Oxford). 2010; 28 (10): 469-72.
2. Koster MI. Making an Epidermis. Annals of the New York Academy of Sciences. 2009; 1170 (1): 7-10.
3. Barcaui Ed O, Carvalho A C P, Piñeiro-Maceira J, Barcaui C B, Moraes H. Study of the skin anatomy with high-frequency (22 MHZ) ultrasonography and histological correlation. Radiologia brasileira. 2015; 48 (5): 324-9.
4. Lai-Cheong J E, McGrath J A. Structure and function of skin, hair and nails. Medicine (Abingdon 1995, UK ed). 2021; 49 (6): 337-42.
5. Tulah A S, Birch-Machin M A. Stressed out mitochondria: The role of mitochondria in ageing and cancer focusing on strategies and opportunities in human skin. Mitochondrion. 2013; 13 (5): 444-53.
6. Birch-Machin M A, Swalwell H. How mitochondria record the effects of UV exposure and oxidative stress using human skin as a model tissue. Mutagenesis. 2009; 25 (2): 101-7.
7. Naidoo K, Hanna R, Birch-Machin MA. What is the role of mitochondrial dysfunction in skin photoaging? Experimental dermatology. 2018; 27 (2): 124-8.
8. Birch-machin M A, Tindall M, Turner R, Haldane F, Rees J L. Mitochondrial DNA Deletions in Human Skin Reflect Photo-Rather Than Chronologic Aging. Journal of investigative dermatology. 1998; 110 (2): 149-52.
9. Wlaschek M, Tantcheva-Poor I, Naderi L, Ma W, Schneider L A, Razi-Wolf Z, et al. Solar UV irradiation and dermal photoaging. Journal of photochemistry and photobiology B, Biology. 2001; 63 (1): 41-51

| SEQUENCES | |
|---|---|
| (2458 bp region of mtDNA susceptible to UVR induced mtDNA damage) | |
| SEQ ID NO: 1 | |
| 4512 | GCAGGCACA CTCATCACAG CGCTAAGCTC GCACTGATTT TTTACCTGAG |
| 4561 | TAGGCCTAGA AATAAACATG CTAGCTTTTA TTCCAGTTCT AACCAAAAAA ATAAACCCTC |
| 4621 | GTTCCACAGA AGCTGCCATC AAGTATTTCC TCACGCAAGC AACCGCATCC ATAATCCTTC |
| 4681 | TAATAGCTAT CCTCTTCAAC AATATACTCT CCGGACAATG AACCATAACC AATACTACCA |
| 4741 | ATCAATACTC ATCATTAATA ATCATAATAG CTATAGCAAT AAAACTAGGA ATAGCCCCCT |
| 4801 | TTCACTTCTG AGTCCCAGAG GTTACCCAAG GCACCCCTCT GACATCCGGC CTGCTTCTTC |
| 4861 | TCACATGACA AAAACTAGCC CCCATCTCAA TCATATACCA AATCTCTCCC TCACTAAACG |
| 4921 | TAAGCCTTCT CCTCACTCTC TCAATCTTAT CCATCATAGC AGGCAGTTGA GGTGGATTAA |
| 4981 | ACCAAACCCA GCTACGCAAA ATCTTAGCAT ACTCCTCAAT TACCCACATA GGATGAATAA |
| 5041 | TAGCAGTTCT ACCGTACAAC CCTAACATAA CCATTCTTAA TTTAACTATT TATATTATCC |
| 5101 | TAACTACTAC CGCATTCCTA CTACTCAACT TAAACTCCAG CACCACGACC CTACTACTAT |
| 5161 | CTCGCACCTG AAACAAGCTA ACATGACTAA CACCCTTAAT TCCATCCACC CTCCTCTCCC |
| 5221 | TAGGAGGCCT GCCCCCGCTA ACCGGCTTTT TGCCCAAATG GGCCATTATC GAAGAATTCA |
| 5281 | CAAAAAACAA TAGCCTCATC ATCCCCACCA TCATAGCCAC CATCACCCTC CTTAACCTCT |
| 5341 | ACTTCTACCT ACGCCTAATC TACTCCACCT CAATCACACT ACTCCCCATA TCTAACAACG |
| 5401 | TAAAAATAAA ATGACAGTTT GAACATACAA AACCCACCCC ATTCCTCCCC ACACTCATCG |
| 5461 | CCCTTACCAC GCTACTCCTA CCTATCTCCC CTTTTATACT AATAATCTTA TAGAAATTTA |
| 5521 | GGTTAAATAC AGACCAAGAG CCTTCAAAGC CCTCAGTAAG TTGCAATACT TAATTTCTGT |
| 5581 | AACAGCTAAG GACTGCAAAA CCCCACTCTG CATCAACTGA ACGCAAATCA GCCACTTTAA |
| 5641 | TTAAGCTAAG CCCTTACTAG ACCAATGGGA CTTAAACCCA CAAACACTTA GTTAACAGCT |
| 5701 | AAGCACCCTA ATCAACTGGC TTCAATCTAC TTCTCCCGCC GCCGGGAAAA AAGGCGGGAG |
| 5761 | AAGCCCCGGC AGGTTTGAAG CTGCTTCTTC GAATTTGCAA TTCAATATGA AAATCACCTC |
| 5821 | GGAGCTGGTA AAAAGAGGCC TAACCCCTGT CTTTAGATTT ACAGTCCAAT GCTTCACTCA |
| 5881 | GCCATTTTAC CTCACCCCCA CTGATGTTCG CCGACCGTTG ACTATTCTCT ACAAACCACA |
| 5941 | AAGACATTGG AACACTATAC CTATTATTCG GOGCATGAGC TGGAGTCCTA GGCACAGCTC |
| 6001 | TAAGCCTCCT TATTCGAGCC GAGCTGGGCC AGCCAGGCAA CCTTCTAGGT AACGACCACA |
| 6061 | TCTACAACGT TATCGTCACA GCCCATGCAT TTGTAATAAT CTTCTTCATA GTAATACCCA |
| 6121 | TCATAATCGG AGGCTTTGGC AACTGACTAG TTCCCCTAAT AATCGGTGCC CCCGATATGG |
| 6181 | CGTTTCCCCG CATAAACAAC ATAAGCTTCT GACTCTTACC TCCCTCTCTC CTACTCCTGC |
| 6241 | TCGCATCTGC TATAGTGGAG GCCGGAGCAG GAACAGGTTG AACAGTCTAC CCTCCCTTAG |
| 6301 | CAGGGAACTA CTCCCACCCT GGAGCCTCCG TAGACCTAAC CATCTTCTCC TTACACCTAG |
| 6361 | CAGGTGTCTC CTCTATCTTA GGGGCCATCA ATTTCATCAC AACAATTATC AATATAAAAC |
| 6421 | CCCCTGCCAT AACCCAATAC CAAACGCCCC TCTTCGTCTG ATCCGTCCTA ATCACAGCAG |
| 6481 | TCCTACTTCT CCTATCTCTC CCAGTCCTAG CTGCTGGCAT CACTATACTA CTAACAGACC |
| 6541 | GCAACCTCAA CACCACCTTC TTCGACCCCG CCGGAGGAGG AGACCCCATT CTATACCAAC |
| 6601 | ACCTATTCTG ATTTTTCGGT CACCCTGAAG TTTATATTCT TATCCTACCA GGCTTCGGAA |
| 6661 | TAATCTCCCA TATTGTAACT TACTACTCCG GAAAAAAAGA ACCATTIGGA TACATAGGTA |
| 6721 | TGGTCTGAGC TATGATATCA ATTGGCTTCC TAGGGTTTAT CGTGTGAGCA CACCATATAT |
| 6781 | TTACAGTAGG AATAGACGTA GACACACGAG CATATTTCAC CTCCGCTACC ATAATCATCG |
| 6841 | CTATCCCCAC CGGCGTCAAA GTATTTAGCT GACTCGCCAC ACTCCACGGA AGCAATATGA |
| 6901 | AATGATCTGC TGCAGTGCTC TGAGCCCTAG GATTCATCTT TCTTTTCACC GTAGGTGGCC |
| 6961 | TGACTGGCA |
| (1233 bp region of mtDNA susceptible to UVR induced mtDNA damage) | |
| SEQ ID NO: 2 | |
| 4512 | GCAGGCACA CTCATCACAG CGCTAAGCTC GCACTGATTT TTTACCTGAG |
| 4561 | TAGGCCTAGA AATAAACATG CTAGCTTTTA TTCCAGTTCT AACCAAAAAA ATAAACCCTC |
| 4621 | GTTCCACAGA AGCTGCCATC AAGTATTTCC TCACGCAAGC AACCGCATCC ATAATCCTTC |
| 4681 | TAATAGCTAT CCTCTTCAAC AATATACTCT CCGGACAATG AACCATAACC AATACTACCA |
| 4741 | ATCAATACTC ATCATTAATA ATCATAATAG CTATAGCAAT AAAACTAGGA ATAGCCCCCT |
| 4801 | TTCACTTCTG AGTCCCAGAG GTTACCCAAG GCACCCCTCT GACATCCGGC CTGCTTCTTC |
| 4861 | TCACATGACA AAAACTAGCC CCCATCTCAA TCATATACCA AATCTCTCCC TCACTAAACG |
| 4921 | TAAGCCTTCT CCTCACTCTC TCAATCTTAT CCATCATAGC AGGCAGTTGA GGTGGATTAA |
| 4981 | ACCAAACCCA GCTACGCAAA ATCTTAGCAT ACTCCTCAAT TACCCACATA GGATGAATAA |
| 5041 | TAGCAGTTCT ACCGTACAAC CCTAACATAA CCATTCTTAA TTTAACTATT TATATTATCC |
| 5101 | TAACTACTAC CGCATTCCTA CTACTCAACT TAAACTCCAG CACCACGACC CTACTACTAT |
| 5161 | CTCGCACCTG AAACAAGCTA ACATGACTAA CACCCTTAAT TCCATCCACC CTCCTCTCCC |
| 5221 | TAGGAGGCCT GCCCCCGCTA ACCGGCTTTT TGCCCAAATG GGCCATTATC GAAGAATTCA |
| 5281 | CAAAAAACAA TAGCCTCATC ATCCCCACCA TCATAGCCAC CATCACCCTC CTTAACCTCT |
| 5341 | ACTTCTACCT ACGCCTAATC TACTCCACCT CAATCACACT ACTCCCCATA TCTAACAACG |
| 5401 | TAAAAATAAA ATGACAGTTT GAACATACAA AACCCACCCC ATTCCTCCCC ACACTCATCG |
| 5461 | CCCTTACCAC GCTACTCCTA CCTATCTCCC CTTTTATACT AATAATOTTA TAGAAATTTA |
| 5521 | GGTTAAATAC AGACCAAGAG CCTTCAAAGC CCTCAGTAAG TTGCAATACT TAATTTCTGT |
| 5581 | AACAGCTAAG GACTGCAAAA CCCCACTCTG CATCAACTGA ACGCAAATCA GCCACTTTAA |
| 5641 | TTAAGCTAAG CCCTTACTAG ACCAATGGGA CTTAAACCCA CAAACACTTA GTTAACAGCT |
| 5701 | AAGCACCCTA ATCAACTGGC TTCAATCTAC TTCTCCCGCC GCCG |
| (1229 bp region of mtDNA susceptible to UVR induced mtDNA damage) | |
| SEQ ID NO: 3 | |
| 5741 | GCCGGGAAAA AAGGCGGGAG |
| 5761 | AAGCCCCGGC AGGTTTGAAG CTGCTTCTTC GAATTTGCAA TTCAATATGA AAATCACCTC |
| 5821 | GGAGCTGGTA AAAAGAGGCC TAACCCCTGT CTTTAGATTT ACAGTCCAAT GCTTCACTCA |
| 5881 | GCCATTTTAC CTCACCCCCA CTGATGTTCG CCGACCGTTG ACTATTCTCT ACAAACCACA |
| 5941 | AAGACATTGG AACACTATAC CTATTATTCG GCGCATGAGC TGGAGTCCTA GGCACAGCTC |
| 6001 | TAAGCCTCCT TATTCGAGCC GAGCTGGGCC AGCCAGGCAA CCTTCTAGGT AACGACCACA |
| 6061 | TCTACAACGT TATCGTCACA GCCCATGCAT TTGTAATAAT CTTCTTCATA GTAATACCCA |
| 6121 | TCATAATCGG AGGCTTTGGC AACTGACTAG TTCCCCTAAT AATCGGTGCC CCCGATATGG |
| 6181 | CGTTTCCCCG CATAAACAAC ATAAGCTTCT GACTCTTACC TCCCTCTCTC CTACTCCTGC |
| 6241 | TCGCATCTGC TATAGTGGAG GCCGGAGCAG GAACAGGTTG AACAGTCTAC CCTCCCTTAG |
| 6301 | CAGGGAACTA CTCCCACCCT GGAGCCTCCG TAGACCTAAC CATCTTCTCC TTACACCTAG |
| 6361 | CAGGTGTCTC CTCTATCTTA GGGGCCATCA ATTTCATCAC AACAATTATC AATATAAAAC |
| 6421 | CCCCTGCCAT AACCCAATAC CAAACGCCCC TCTTCGTCTG ATCCGTCCTA ATCACAGCAG |
| 6481 | TCCTACTTCT CCTATCTCTC CCAGTCCTAG CTGCTGGCAT CACTATACTA CTAACAGACC |
| 6541 | GCAACCTCAA CACCACCTTC TTCGACCCCG CCGGAGGAGG AGACCCCATT CTATACCAAC |
| 6601 | ACCTATTCTG ATTTTTCGGT CACCCTGAAG TTTATATTCT TATCCTACCA GGCTTCGGAA |
| 6661 | TAATCTCCCA TATTGTAACT TACTACTCCG GAAAAAAAGA ACCATTIGGA TACATAGGTA |
| 6721 | TGGTCTGAGC TATGATATCA ATTGGCTTCC TAGGGTTTAT CGTGTGAGCA CACCATATAT |
| 6781 | TTACAGTAGG AATAGACGTA GACACACGAG CATATTTCAC CTCCGCTACC ATAATCATCG |
| 6841 | CTATCCCCAC CGGCGTCAAA GTATTTAGCT GACTCGCCAC ACTCCACGGA AGCAATATGA |
| 6901 | AATGATCTGC TGCAGTGCTC TGAGCCCTAG GATTCATCTT TCTTTTCACC GTAGGTGGCC |
| 6961 | TGACTGGCA |
| (218 bp region of mtDNA susceptible to UVR induced mtDNA damage) | |
| SEQ ID NO: 16 | |
| 6067 | ACGT TATCGTCACA GCCCATGCAT TTGTAATAAT CTTCTTCATA GTAATACCCA |
| 6121 | TCATAATCGG AGGCTTTGGC AACTGACTAG TTCCCCTAAT AATCGGTGCC CCCGATATGG |
| 6181 | CGTTTCCCCG CATAAACAAC ATAAGCTTCT GACTCTTACC TCCCTCTCTC CTACTCCTGC |
| 6241 | TCGCATCTGC TATAGTGGAG GCCGGAGCAG GAACAGGTTG AACA |
| (495 bp region of mtDNA susceptible to UVR induced mtDNA damage) | |
| SEQ ID NO: 17 | |
| 6067 | ACGT TATCGTCACA GCCCATGCAT TTGTAATAAT CTTCTTCATA GTAATACCCA |
| 6121 | TCATAATCGG AGGCTTTGGC AACTGACTAG TTCCCCTAAT AATCGGTGCC CCCGATATGG |
| 6181 | CGTTTCCCCG CATAAACAAC ATAAGCTTCT GACTCTTACC TCCCTCTCTC CTACTCCTGC |
| 6241 | TCGCATCTGC TATAGTGGAG GCCGGAGCAG GAACAGGTTG AACAGTCTAC CCTCCCTTAG |
| 6301 | CAGGGAACTA CTCCCACCCT GGAGCCTCCG TAGACCTAAC CATCTTCTCC TTACACCTAG |
| 6361 | CAGGTGTCTC CTCTATCTTA GGGGCCATCA ATTTCATCAC AACAATTATC AATATAAAAC |
| 6421 | CCCCTGCCAT AACCCAATAC CAAACGCCCC TCTTCGTCTG ATCCGTCCTA ATCACAGCAG |
| 6481 | TCCTACTTCT CCTATCTCTC CCAGTCCTAG CTGCTGGCAT CACTATACTA CTAACAGACC |
| 6541 | GCAACCTCAA CACCACCTTC T |
| (648 bp region of mtDNA susceptible to UVR induced mtDNA damage) | |
| SEQ ID NO: 18 | |
| 4619 | TC |
| 4621 | GTTCCACAGA AGCTGCCATC AAGTATTTCC TCACGCAAGC AACCGCATCC ATAATCCTTC |
| 4681 | TAATAGCTAT CCTCTTCAAC AATATACTCT CCGGACAATG AACCATAACC AATACTACCA |
| 4741 | ATCAATACTC ATCATTAATA ATCATAATAG CTATAGCAAT AAAACTAGGA ATAGCCCCCT |
| 4801 | TTCACTTCTG AGTCCCAGAG GTTACCCAAG GCACCCCTCT GACATCCGGC CTGCTTCTTC |
| 4861 | TCACATGACA AAAACTAGCC CCCATCTCAA TCATATACCA AATCTCTCCC TCACTAAACG |
| 4921 | TAAGCCTTCT CCTCACTCTC TCAATCTTAT CCATCATAGC AGGCAGTTGA GGTGGATTAA |
| 4981 | ACCAAACCCA GCTACGCAAA ATCTTAGCAT ACTCCTCAAT TACCCACATA GGATGAATAA |
| 5041 | TAGCAGTTCT ACCGTACAAC CCTAACATAA CCATTCTTAA TTTAACTATT TATATTATCC |
| 5101 | TAACTACTAC CGCATTCCTA CTACTCAACT TAAACTCCAG CACCACGACC CTACTACTAT |
| 5161 | CTCGCACCTG AAACAAGCTA ACATGACTAA CACCCTTAAT TCCATCCACC CTCCTCTCCC |
| 5221 | TAGGAGGCCT GCCCCCGCTA ACCGGCTTTT TGCCCAAATG GGCCAT |

1. A method of determining the level of mitochondrial DNA (mtDNA) damage in a cell population, the method comprising:
a) quantifying the total amount of mtDNA in a sample of the cell population;
b) quantifying the amount of a mtDNA fragment comprising at least 200 bases from a region of mtDNA located between nucleotides from 4412 to 7069 in the sample of the cell population; and
c) comparing the amount of said fragment to the total amount of mtDNA in the sample, and thereby determining the level of mtDNA damage in the cell population.

2. A method of determining the ability of a test agent to prevent or repair mtDNA damage in a cell population, the method comprising:
a) determining the level of mtDNA damage in a first sample of the cell population by a method according to claim 1;
b) providing the test agent to the cell population;
c) determining the level of mtDNA damage in a second sample of the cell population by a method according to claim 1; and
d) comparing the level of mtDNA damage determined in step c) to the levels of mtDNA damage determined in step a); wherein:
i) no change between the level of mtDNA damage determined in step c) as compared to step a) is indicative of the test agent having the ability to prevent mtDNA damage; or
ii) a decrease between the level of mtDNA damage determined in step c) as compared to step a) is indicative of the test agent having the ability to repair mtDNA damage.

3. A method of monitoring progression of mtDNA damage in a cell population, the method comprising:
a) determining the level of mtDNA damage in a first sample of the cell population by a method according to claim 1;
b) determining the level of mtDNA damage in a second sample of the cell population by a method according to claim 1, wherein the second sample has been obtained from the cell population at a later time point than the first sample; and
c) comparing the amount of mtDNA damage determined in step b) as compared to step a); wherein an increase in mtDNA damage in step b) as compared to step a) is indicative of progression of mtDNA damage.

4. A kit for determining the level of mitochondrial DNA (mtDNA) damage in a cell population comprising a primer set for amplifying a mtDNA fragment comprising at least about 200 bases from a region of mtDNA located between nucleotides from 4412 to 7069.

5. The method of any one of claims 1 to 3, or the kit of claim 4, wherein the cell population is a skin cell population.

6. The method of any one of claims 1 to 5, or the kit claim 4 or 5, wherein the mtDNA damage is caused by oxidative stress.

7. The method or kit of claim 6, wherein the oxidative stress is caused by exposure to UVR exposure and/or a pollutant, optionally wherein the pollutant is urban dust.

8. The method of any one of claims 1 to 3 or 5 to 7, or the kit of any one of claims 4 to 7, wherein the fragment is from a region of mtDNA located between nucleotides from 4512 to 6969.

9. The method of any one of claims 1 to 3 or 5 to 8, or the kit of any one of claims 4 to 8, wherein the fragment comprises at least about 200 bases.

10. The method or kit of claim 9, wherein the fragment comprises at least about 500 bases.

11. The method or kit of claim 10, wherein the fragment comprises at least about 650 bases.

12. The method or kit of claim 11, wherein the fragment comprises at least about 1000 bases.

13. The method or kit of claim 12, wherein the fragment comprises at least about 1200 bases.

14. The method or kit of claim 13, wherein the fragment comprises at least 2000 bases.

15. The method or kit of claim 13, wherein the fragment comprises at least about 2400 bases, optionally wherein the fragment comprises or consists of 2457 bases.

16. The method or kit of claim 7, wherein the exposure to UVR is chronic or wherein exposure to pollution is acute.

17. The method or kit of claim 16, wherein the fragment is from a region of mtDNA located between nucleotides from 4512 to 5744.

18. The method or kit of claim 17, wherein the fragment is at least about 200 bases.

19. The method or kit of claim 18, wherein the fragment is at least about 500 bases.

20. The method or kit of claim 19, wherein the fragment is at least about 650 bases.

21. The method or kit of claim 20, wherein the fragment is at least about 1000 bases.

22. The method or kit of claim 21, wherein the fragment comprises at least about 1200 bases, optionally wherein the fragment comprises or consists of 1233 bases.

23. The method or kit of claim 7, wherein the exposure to UVR is acute or wherein exposure to pollution is chronic.

24. The method or kit of claim 23, wherein the fragment is from a region of mtDNA located between nucleotides from 5741 to 6969.

25. The method or kit of claim 24, wherein the fragment is at least about 200 bases.

26. The method or kit of claim 25, wherein the fragment is at least about 500 bases.

27. The method or kit of claim 26, wherein the fragment is at least about 650 bases.

28. The method or kit of claim 27, wherein the fragment is at least about 1000 bases.

29. The method or kit of claim 28, wherein the fragment comprises at least about 1200 bases, optionally wherein the fragment comprises or consists of 1229 bases.

30. The method of any one of claims 1 to 3 or 5 to 29, wherein the step of quantifying the total amount of mtDNA comprises amplifying a damage resistant mtDNA region.

31. The method of claim 30, wherein the damage resistant mtDNA region is a fragment consisting of about 100 bases or less.

32. The method of claim 30, wherein the damage resistant region is a fragment consisting of 83 bases or less, optionally located between nucleotides from 16042 to 16124.

33. The method of any one of the preceding claims, wherein the step of quantifying the amount of a mtDNA fragment comprises amplifying the fragment.

34. The method of any of one claims 29 to 32, wherein amplifying is by quantitative PCR (qPCR).

35. The kit of any one of claims 4 to 28, wherein the primer set comprises nucleic acid sequences selected from the group consisting of (1) SEQ ID NO: 4 and SEQ ID NO: 7; (2) SEQ ID NO: 4 and SEQ ID NO: 5; (3) SEQ ID NO: 6 and SEQ ID NO: 7; (4) SEQ ID NO: 10 and SEQ ID NO: 11; (5) SEQ ID NO: 12 and SEQ ID NO: 13; and (5) SEQ ID NO: 14 and SEQ ID NO: 15.

36. The kit of claim 35, wherein the kit comprises a primer set for amplifying a damage resistant region of mtDNA, optionally wherein the primer set comprises the nucleic acid sequences according to SEQ ID NO: 8 and SEQ ID NO:9.

37. A use of a mtDNA fragment comprising at least about 200 bases from a region of mtDNA located between nucleotides from 4412 to 7069 for determining mtDNA damage.

38. The use of a mtDNA fragment according to claim 37, comprising at least about 200 bases from a region of mtDNA located between nucleotides from 4512 to 6969.


SEQUENCE LISTING (18 sequences)

SEQ ID NO: 1 - DNA, 2458 bp, Homo sapiens
  source 1..2458: mol_type = genomic DNA
gcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggca

SEQ ID NO: 2 - DNA, 1233 bp, Homo sapiens
  source 1..1233: mol_type = genomic DNA
gcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccg

SEQ ID NO: 3 - DNA, 1229 bp, Homo sapiens
  source 1..1229: mol_type = genomic DNA
gccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggca

SEQ ID NO: 4 - DNA, 21 bp, synthetic construct
  source 1..21: mol_type = other DNA
  misc_feature 1..21: note = Forward primer 6F
gcaggcacactcatcacagcg

SEQ ID NO: 5 - DNA, 22 bp, synthetic construct
  source 1..22: mol_type = other DNA
  misc_feature 1..22: note = Reverse primer 6R
cggcggcgggagaagtagattg

SEQ ID NO: 6 - DNA, 20 bp, synthetic construct
  source 1..20: mol_type = other DNA
  misc_feature 1..20: note = Forward primer 7F
gccgggaaaaaaggcgggag

SEQ ID NO: 7 - DNA, 20 bp, synthetic construct
  source 1..20: mol_type = other DNA
  misc_feature 1..20: note = Reverse primer 7R
tgccagtcaggccacctacg

SEQ ID NO: 8 - DNA, 24 bp, synthetic construct
  source 1..24: mol_type = other DNA
  misc_feature 1..24: note = Forward primer IS1
gatttgggtaccacccaagtattg

SEQ ID NO: 9 - DNA, 23 bp, synthetic construct
  source 1..23: mol_type = other DNA
  misc_feature 1..23: note = Reverse primer IS2
aatattcatggtggctggcagta

SEQ ID NO: 10 - DNA, 22 bp, synthetic construct
  source 1..22: mol_type = other DNA
  misc_feature 1..22: note = Forward primer 218bp
acgttatcgtcacagcccatgc

SEQ ID NO: 11 - DNA, 22 bp, synthetic construct
  source 1..22: mol_type = other DNA
  misc_feature 1..22: note = Reverse primer 218bp
tgttcaacctgttcctgctccg

SEQ ID NO: 12 - DNA, 22 bp, synthetic construct
  source 1..22: mol_type = other DNA
  misc_feature 1..22: note = Forward primer 495bp
acgttatcgtcacagcccatgc

SEQ ID NO: 13 - DNA, 22 bp, synthetic construct
  source 1..22: mol_type = other DNA
  misc_feature 1..22: note = Reverse primer 495bp
agaaggtggtgttgaggttgcg

SEQ ID NO: 14 - DNA, 22 bp, synthetic construct
  source 1..22: mol_type = other DNA
  misc_feature 1..22: note = Forward primer 648bp
tcgttccacagaagctgccatc

SEQ ID NO: 15 - DNA, 22 bp, synthetic construct
  source 1..22: mol_type = other DNA
  misc_feature 1..22: note = Reverse primer 648bp
atggcccatttgggcaaaaagc

SEQ ID NO: 16 - DNA, 218 bp, Homo sapiens
  source 1..218: mol_type = genomic DNA
acgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaaca

SEQ ID NO: 17 - DNA, 495 bp, Homo sapiens
  source 1..495: mol_type = genomic DNA
acgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttct

SEQ ID NO: 18 - DNA, 648 bp, Homo sapiens
  source 1..648: mol_type = genomic DNA
tcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccat